(19) United States
(12) Patent Application Publication
(10) Pub. No.: US 2023/0346810 A1
(43) Pub. Date: Nov. 2, 2023

(54) ENDOGENOUS STABILIZATION OF CANNABINOIDS

(71) Applicant: Abstrax Tech Inc., Tustin, CA (US)

(72) Inventors: Iain W.H. OSWALD, Tustin, CA (US); Twinkle R. PARYANI, Tustin, CA (US); Marcos A. OJEDA, Tustin, CA (US); Manuel E. SOSA HERNANDEZ, Tustin, CA (US); Kevin A. KOBY, Tustin, CA (US); Thomas J. MARTIN, JR., Tustin, CA (US)

(21) Appl. No.: 18/308,148

(22) Filed: Apr. 27, 2023

Related U.S. Application Data
(60) Provisional application No. 63/336,710, filed on Apr. 29, 2022.
(60) Provisional application No. 63/359,111, filed on Jul. 7, 2022.

(51) Int. Cl. A61K 31/00; A61K 47/12

(52) U.S. Cl. CPC A61K 31/658; A61K 47/12

(57) ABSTRACT

A method for making a stabilized cannabinoid composition includes combining an active cannabinoid, a cannabinoic acid and optionally a carrier to provide a product, wherein the acid is present in an amount effective to improve the stability of the composition.

### CROSS REFERENCE TO RELATED APPLICATIONS

This application claims the benefit of U.S. Provisional Application No. 62/336,710, filed Apr. 29, 2022, and U.S. Provisional Application No. 63/359,111, filed Jul. 7, 2022, the disclosures of each of which are incorporated herein by reference in their entirety.

### FIELD

The present technology is generally related to cannabinoids. More specifically, it is related to stabilizing high purity cannabinoids against oxidation effects and color changes.

### BACKGROUND

Cannabis consumption has increased substantially over the last decade due to legalization across the United States and other countries. This has led to numerous types of cannabis-based products to be developed that can be consumed in different fashions. For instance, portable cannabis vaporizer devices, colloquially known as “vapes”, have rapidly proliferated into the marketplace due to their discreetness, ease of use, and lack of combustion compared to traditionally smoked flower. These products rely on a heating a cannabis-derived oil to high temperatures to vaporize directly into the user's lungs. In many cases, this process relies on isolating a single cannabinoid such as THC or CBD in very high potency that is then further mixed with flavoring agents such as terpenes.

In recent years, very high purity cannabinoid (>90%) oils have been isolated and are available commercially. These products include D9-THC, D8-THC, CBD, and other less commonly used cannabinoids. Additionally, Cannabis biosynthesizes over 100 known cannabinoids with important commercial and medicinal value. These compounds include tetrahydrocannabinolic acid (THCA), Cannabidiolic acid (CBDA),

Cannabigerolic acid (CBGA), and myriad others. These compounds are typically decarboxylated into their active, non-acidic analogues during processing. Recent progress in optimizing this process has resulted in very high purity extracts over 98%. These extracts are often further formulated with a variety of other additives depending on the final application of the product. However, high purity non-acidic or active cannabinoids are prone to oxidation in air, especially at temperatures necessary to formulate them into a final product. The present inventors have recently shown that this oxidation can be greatly reduced by incorporating acidic cannabinoids to the extract. However, the high lattice energy of acidic cannabinoids induced by the hydrogen bonding between neighboring molecules hinders their easy incorporation into the viscous oils of the active cannabinoids. It would be advantageous to develop methods to integrate acidic cannabinoids into the viscous active cannabinoid media, which can be used at various stages of production depending on the infrastructure and techniques used to produce them. Additionally, there is a real need for a more cost-effective method of reducing oxidation of high purity active cannabinoids. The inventions described herein meet that long-felt need.

### SUMMARY

In one aspect, a method for making a stabilized cannabinoid composition is provided, wherein the method includes combining an active cannabinoid, a cannabinoic acid and optionally a carrier to provide a product; wherein the acid is present in an amount effective to improve the stability of the composition.

In any embodiment, the active cannabinoid may include, but is not limited to, tetrahydrocannabinol (THC). In any embodiment, the active cannabinoid may include, but is not limited to, tetrahydrocannabinol (THC), cannabidiol (CBD), cannabinol (CBN), cannabichromene (CBC), cannabigerol (CBG), cannabinodiol (CBND), cannabicyclol (CBL), hypocannabinol (CBV), tetrahydrocannabivarin (THCV), hypocannabinol phenol (CBDV), cannabichromevarin (CBCV), cannabigerovarin (CBGV), cannabichromenic acid monomethyl ether (CBGM), or a combination of any two or more thereof In any embodiment, the cannabinoic acid is an endogenous cannabinoic acid. In any embodiment, the endogenous cannabinoic acid may include tetrahydrocannabinolic acid (THCA). In any embodiment, the endogenous cannabinoic acid may include tetrahydrocannabinolic acid (THCA), cannabidiolic acid (CBDA), cannabinolic acid (CBNA), cannabichromenic acid (CBCA), cannabigerolic Acid (CBGA), cannabinodiolic acid (CBNDA), cannabicyclolic acid (CBLA), cannabivarinic acid (CBVA), tetrahydrocannabivarinic acid (THCVA), cannabidivarinic acid (CBDVA), cannabichrovarinic acid (CBCVA), cannabigerovarinic acid (CBGVA), cannabigerol monomethylether (CBGM), cannabigerolic acid monomethylether (CBGAM), and a combination of any two or more thereof.

In any embodiment, the composition includes a carrier such as, but not limited to, methanol, ethanol, isopropanol, butanol, propane, butane, pentane, hexane, heptane, petroleum ether, methyl tertbutyl ether, diethyl ether, carbon dioxide, olive oil, vegetable oil, α-bisabolol, lauric acid, myristic acid, palmitic acid, stearic acid, a terpene or a combination of two or more thereof. In some embodiments, the carrier may be triacetin, triethyl citrate, or other common diluents.

In any embodiment, the composition includes from about 50 wt. % to about 99 wt. % of the active cannabinoid and about 1 wt. % to about 50 wt. % of the cannabinoic acid. In any embodiment, the active cannabinoid: cannabinoic acid ratio ranges from 10:1 to 1:1 by weight. In any embodiment, the active cannabinoid has a purity of greater than 90%. In any embodiment, the cannabinoic acid has a purity of greater than 90%.

In any embodiment, the method for making a stabilized cannabinoid composition includes adding the solid cannabinoic acid to the active cannabinoid to provide a mixture, heating the mixture to provide the cannabinoid composition, and cooling the cannabinoid composition.

In any embodiment, the method for making a stabilized cannabinoid composition includes heating the cannabinoic acid to a temperature sufficient to melt the cannabinoid acid, and adding the melt in to the active cannabinoid. In any embodiment, the temperature ranges from 60° C. to 150° C. In some embodiments, the method includes melting the cannabinoic acid, and solvating it into the active cannabinoid.

In any embodiment, the method for making a stabilized cannabinoid composition may include heating the cannabinoic acid to a temperature sufficient to provide a glass-like material, and adding the glass-like material in to the active cannabinoid. In any embodiment, the temperature may be from from about 80° C. to about 200° C. In any embodiment, the composition may include greater than about 20 wt. % cannabinoic acid.

In any embodiment, the method for making a stabilized cannabinoid composition may include solubilizing the cannabinoic acid in a carrier to provide a solution, and adding the solution to the active cannabinoid. In any embodiment, the carrier may include a flavor modifier or fragrance modifier. In any embodiment, the flavor modifier may include one or more terpenes or a terpene blend.

In one aspect, provided is a composition that includes a cannabinoic acid and active cannabinoid. In any embodiment, the composition may include about 1 wt. % to about 60 wt. % of a cannabinoic acid, and about 40 wt. % to about 99 wt. % of active cannabinoid, and less than 2 wt. % of endogenous terpenes. In any embodiment, the composition may also include about 1 wt. % to about 98 wt. % of a carrier. In any embodiment, the composition may further include a flavor modifier or a fragrance modifier. In any embodiment, the flavor modifier may include one or more terpenes.

In one aspect, provided is a composition that includes about 4 wt. % to about 15 wt. % of a cannabinoic acid, about 40 wt. % to about 99 wt. % of active cannabinoid and about 4 wt. % to about 15 wt. % of one or more terpenes. In any embodiment, the one or more terpenes are exogenously added terpenes.

In any embodiment, the active cannabinoid may be Δ⁸-tetrahydrocannabinol (Δ⁸-THC), Δ⁹-tetrahydrocannabinol (Δ9-THC), Δ¹⁰-tetrahydrocannabinol (Δ¹⁰-THC), cannabidiol, or a combination of any two or more thereof. In any embodiment, the cannabinoic acid may include tetrahydrocannabinolic acid (THCA), cannabidiolic acid (CBDA), cannabinolic acid (CBNA), cannabichromenic acid (CBCA), cannabigerolic acid (CBGA), cannabinodiolic acid (CBNDA), cannabicyclolic acid (CBLA), cannabivarinic acid (CBVA), tetrahydrocannabivarinic acid (THCVA), cannabidivarinic acid (CBDVA), cannabichrovarinic acid (CBCVA), cannabigerovarinic acid (CBGVA), cannabigerol monomethylether (CBGM), cannabigerolic acid monomethylether (CBGAM), and a combination of any two or more thereof.

In one aspect, a process is provided for preparing a stabilized cannabinoid composition. The process may include providing an active cannabinoid at a purity of greater than 90 wt. %, greater than 95% or greater than 98%, and adding to the cannabinoid a cannabinoic acid at about 3 to about 10 wt. %. As used herein, the active cannabinoid of high purity may be a mixture of active cannabinoids, as long as it is those compounds in high-purity.

In another aspect, a process is provided for preparing a stabilized cannabinoid composition. The process may include terminating decarboxylation of a cannabinoid precursor prior to full conversion, such that a composition contains an active cannabinoid from about 50 wt. % to about 99.5 wt. %, and a cannabinoic acid at about 50 wt. % to about 0.5 wt. %. In some embodiments, the composition may include the active cannabinoid from about 90 wt. % to about 97 wt. %, and the cannabinoic acid at about 10 wt. % to about 3 wt. %.

In another aspect, a stabilized cannabinoid composition may include about 3 wt. % to about 25 wt. % of an cannabinoic acid, and a high purity active cannabinoid.

### BRIEF DESCRIPTION OF THE DRAWINGS

FIGS. 1A and 1B are images showing a control sample, C1, with 0% THCA showing significant discoloration (1A), and an experimental sample, E1, with about 5.9 wt. % THCA showing minimal color change (1B), each following 30 min of exposure to air.

FIGS. 2A and 2B are images showing warm compositions of each of C2 and E2, prepared according to the examples.

FIG. 3 shows the time dependence of samples E2 and C2 samples exposed to air, prepared according to the examples.

FIG. 4 shows images of samples C2 and E2 in duplicate after slow cooling to room temperature in air for 24 hours.

FIG. 5 shows images of samples C2 and E2 in duplicate after being homogenized with botanical terpenes at high temperatures and after cooling.

FIG. 6 shows images of samples CBD-C1 having no CBDA and CBD-E1 having 5% CBDA exposed to air, prepared according to the examples.

FIG. 7 is a plot of normalized absorbance (arbitrary units or arb. units) against wavelength (nanometers or nm) showing the ultraviolet-visible spectra of samples E1, C1 and CBDA-E1, prepared according to the examples.

FIG. 8 is a plot of normalized absorbance (arbitrary units or arb. units) against wavelength (nanometers or nm) showing the ultraviolet-visible spectra of samples containing D8-THC and D9-THC as the non-acidic cannabinoid and CBDA as the acidic cannabinoid.

FIG. 9 is a plot of normalized absorbance (arbitrary units or arb. units) against wavelength (nanometers or nm) showing the ultraviolet-visible spectra of samples containing a commercially available THC distilled oil sample with 0% THCA, and the THC oil of the invention, with about 5% THCA, with and without the addition of 5% KOH solution.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As utilized herein with respect to numerical ranges, the terms “approximately,” “about,” “substantially,” and similar terms will be understood by persons of ordinary skill in the art and will vary to some extent depending upon the context in which it is used. If there are uses of the terms that are not clear to persons of ordinary skill in the art, given the context in which it is used, the terms will be plus or minus 10% of the disclosed values. When “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The term “high purity” as applied to various materials indicates that the material is present at greater than about 90%, greater than about 91%, greater than about 92%, greater than about 93%, greater than about 94%, greater than about 95%, greater than about 96%, greater than about 97%, greater than about 98%, or greater than about 99%, concentration by weight.

The term “active cannabinoid” or “cannabinoic oil” as used herein in the context of describing various cannabinoid materials, relate to non-acid group containing cannabinoids, neutral cannabinoids, or decarboxylated cannabinoids, for example, tetrahydrocannabinol (THC), cannabidiol (CBD), cannabinol (CBN), cannabichromene (CBC), cannabigerol (CBG), cannabinodiol (CBND), cannabicyclol (CBL), hypocannabinol (CBV), Tetrahydrocannabivarin (THCV), hypocannabinol Phenol (CBDV), Cannabichromevarin (CBCV), Cannabigerovarin (CBGV), Cannabichromenic acid monomethyl ether (CBGM), and the like.

The terms “cannabinoic acid,” “cannabinoid acid,” and “acidic cannabinoid” are used interchangeably and as used herein in the context of describing various cannabinoid materials relate to acid forms of cannabinoids or acid group containing cannabinoid, cannabinoid precursors, or non-decarboxylated cannabinoids, for example, tetrahydrocannabinolic acid (THCA), cannabidiolic acid (CBDA), cannabinolic acid (CBNA), cannabichromenic acid (CBCA), cannabigerolic Acid (CBGA), cannabinodiolic acid (CBNDA), cannabicyclolic acid (CBLA), cannabivarinic acid (CBVA), tetrahydrocannabivarinic acid (THCVA), cannabidivarinic acid (CBDVA), cannabichrovarinic acid (CBCVA), cannabigerovarinic acid (CBGVA), cannabigerol monomethylether (CBGM), cannabigerolic acid monomethylether (CBGAM), and the like.

The term “endogenous” as used herein refers to a component which is present and/or naturally expressed within a plant or a cell thereof. As used herein, the term “exogenous” refers to a component which may not be present and/or naturally expressed within the plant or cell thereof, e.g., a component which may be externally added.

Cannabis consumption has increased substantially over the last decade due to legalization across the United States and other countries. This has led to numerous types of cannabis-based products to be developed that can be consumed in different fashions. For instance, portable cannabis vaporizer devices, colloquially known as “vapes,” have rapidly proliferated into the marketplace due to their discreetness, ease of use, and lack of combustion compared to traditionally smoked flower. These products rely on a heating a cannabis-derived oil, typically rich in tetrahydrocannabinol (THC), to high temperatures to vaporize directly into the user's lungs. The oil used in these products is typically made by extracting the important phytochemicals, such as cannabinoids, terpenes, volatile sulfur compounds, and other secondary metabolites, using a solvent at low temperature. However, preventing oxidation of the active Cannabinoid, especially when it is in a high purity form, is an issue that has been plaguing the industry, and there is very little knowledge and understanding about how to prevent it.

Cannabinoid is a generic term for phytocannabinoids, endocannabinoids, and synthetic cannabinoids. Phytocannabinoids are the natural compounds found in the Cannabis plants. Cannabis biosynthesizes over 100 known cannabinoids including the compound tetrahydrocannabinolic acid (THCA), the precursor to tetrahydrocannabinol (THC), its primary psychoactive ingredient. These compounds are often extracted using hydrocarbon solvents such as butane to yield various types of cannabis extracts. In particular, high purity THCA can be isolated as microcrystals or as large grain crystalline solids in purities over 99 wt. %. These components may then be further converted into the active, liquid compound THC via thermal decarboxylation of THCA. Nonetheless, a common by-product of this chemical change is the formation of Δ9-tetrahydrocannabinol quinone (D9-THCQ) and potentially other byproducts that form when high purity THC is exposed to the oxygen in air. While this reaction is pervasive for THC and other cannabinoids, a means to preventing this chemical process from occurring without exogenous, non-cannabis derived additives has not been established. Provided herein is an effective method for stabilizing high purity THC and other cannabinoids through inclusion of a small percentage of THCA in a purified THC product. The compositions and methods described herein do not require or are free of any exogenous acids (e.g., mineral acids such as phosphoric acid or organic acids, such as citric acid), bases, or other antioxidant products, and instead use materials that are endogenous to cannabis. The process and compositions significantly reduce undesirable color changes in air, and avoid the use of anti-oxidant additives that may pose an inhalation risk to a user or alter the flavor profiles of the product.

Cannabis oil use in vapes has increased substantially in recent years, each defined by ingredients they are composed of or their production process. For instance, early forms of cannabis vaporizers were composed primarily of distilled THC—referred to as “distillate.” In this case, THC is typically produced by decarboxylating THCA into THC using a distillation apparatus. This process can lead to oils with high THC purity (>99%) and minimal color or flavor. Other types of oils include high terpene extracts (HTEs), which are the liquid fraction obtained from the unrefined oil after allowing THCA to crystallize and precipitate out of solution. In this case, THCA is physically separated from the remaining liquid fraction that can then be used as an oil for a vaporization device or other applications.

Recently, high purity THCA (>98%) has been isolated and is available in the marketplace. While this crystalline material may be consumed readily, it may be further processed to create THC via thermal decarboxylation under inert conditions and then used as a substitute to traditional distillate. As this is the liquid product of this process, it is referred to as “liquid diamonds” within the marketplace. The major difference between distillate derived THC and liquid diamonds is the THCA source. In typical distillation, low quality THCA sources can be used to then decarboxylate into THC via high temperature distillation. In this process, THC distillate is created via taking a crude cannabis THCA extract, winterizing, and filtering to increase THCA purity, evaporation of solvent, decarboxylating under heat, and then distilling multiple times to remove any undesirable chemicals including flavorants, residual solvents, etc. While the purity can be very high for these products, the possibility of residual plant material or other undesired secondary metabolites exists due to the initial quality of the plant material typically used. On the other hand, THCA can easily be separated via crystallization when extracted under the correct operating conditions and from plant material that is of sufficient quality. As such, liquid diamonds derived from this source can have less latent phytochemicals remaining in the decarboxylated oil, leading to a more highly desired product with less color or flavor.

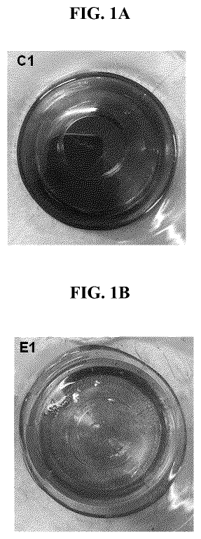

Nonetheless, issues exist with the current technologies for distillate, liquid diamonds, as well as other high purity active (i.e. non-acidic or decarboxylated) cannabinoids. A major obstacle in their production is the formation of oxygen-derived by-products wherein the aromatic alcohol of the cannabinoid of interest is oxidized to form quinones, and other unwanted byproducts. In particular, the compound Δ9-tetrahydrocannabinolquinone (D9-THCQ), and other byproducts, can be produced rapidly when high purity THC is exposed to oxygen as shown in FIGS. 1A and 1B. Likewise, Δ8-tetrahydrocannabinolquinone (D8-THCQ) and cannabidiolquinone (CBDQ) can form in similar conditions from their respective precursors, Δ8-tetrahydrocannabinol (D8-THC) and cannabidiol (CBD). These oxidized compounds, and related byproducts, are often easily identified due to their vibrant red colors. Further, this process is accelerated at higher temperatures necessary to process these oils during formulation or packaging. While inert atmospheres such as nitrogen or argon can prevent oxidation, these oils are near universally exposed to air during processing or in the end use packaging such as in vape carts which inevitably results in oxidation—and color changes—over time. Thus, these high potency products require a means to reduce or prevent oxidation to both enhance the visual quality of the product, as well as ensure it is safe for inhalation. As the inhalation risk of these quinone-containing compounds and other byproducts are not well understood, they not only pose possible unexpected health risks, but also negatively affect the visual quality of cannabis products. While addition of traditional antioxidants such as those used in the food industry or flavor industry may prevent oxidation, possible health risks associated with inhaling these compounds confounds their use. Scheme 1 illustrates the decomposition reaction from THCA (2-Δ9-tetrahydrocannbinolic acid) to THC followed by oxidation into THCQ.

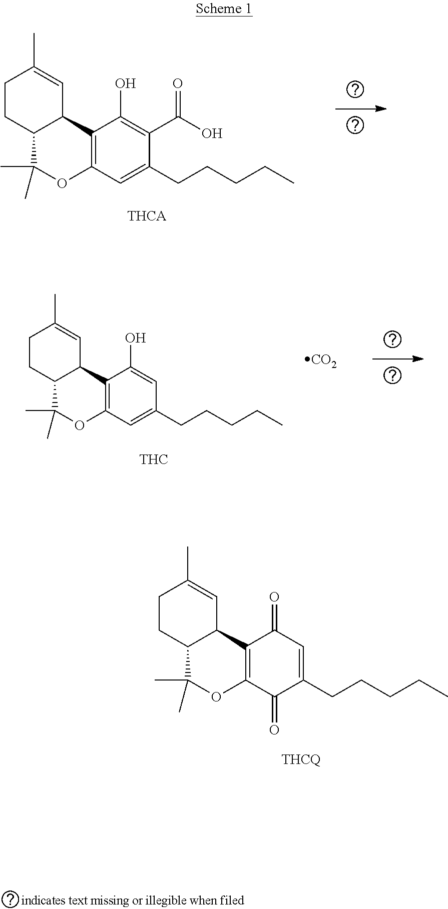

The compositions and methods described herein address the above drawbacks and reduce the formation of the quinone-containing compounds and other possible byproducts e.g., in high-purity active (non-acidic) cannabis extracts without addition of non-cannabis derived ingredients (e.g., organic acids or other exogenous acids). In some embodiments, the compositions and methods of the present technology are free of non-cannabis derived acids e.g., citric acid, ascorbic acid, acetic acid, malic acid, oxalic acid, succinic acid, fumaric acid, salicylic acid, tartaric acid, lactic acid, salicylic acid, carbonic acid, phosphoric acid and the like.

In one aspect, provided herein are stable compositions that may include one or more active cannabinoids, and one or more cannabinoic acids. Active cannabinoids or cannabinoic oils suitable for use in the present technology are well known, and may include non-acid group containing cannabinoids, neutral cannabinoids, or decarboxylated cannabinoids as well as derivatives thereof. Suitable active cannabinoids include, without limitation, tetrahydrocannabinol (THC), cannabidiol (CBD), cannabinol (CBN), cannabichromene (CBC), cannabigerol (CBG), cannabinodiol (CBND), cannabicyclol (CBL), hypocannabinol (CBV), Tetrahydrocannabivarin (THCV), hypocannabinol phenol (CBDV), cannabichromevarin (CBCV), cannabigerovarin (CBGV), cannabichromenic acid monomethyl ether (CBGM), and the like or derivatives thereof, or a combination of any two or more thereof. The active cannabinoid may include both synthetic cannabinoids (e.g., d8-THC) as well as endogenous cannabinoids (e.g., d9-THC).

The active cannabinoid and/or the cannabinoic acid of the present compositions may include high purity active cannabinoid and/or high purity cannabinoic acid. For example, high purity active cannabinoid may include greater than about 90%, greater than about 95%, greater than about 96%, greater than about 97%, greater than about 98%, or greater than about 99% of the active cannabinoid. Likewise, high purity cannabinoid acid (e.g., THCA) may include greater than about 90%, greater than about 95%, greater than about 96%, greater than about 97%, greater than about 98%, or greater than about 99% of the cannabinoic acid. In some embodiments, the active cannabinoid is THC having a purity greater than about 90%, greater than about 95%, greater than about 96%, greater than about 97%, greater than about 98%, or greater than about 99%. In some embodiments, the cannabinoid acid is THCA having a purity greater than about 90%, greater than about 95%, greater than about 96%, greater than about 97%, greater than about 98%, or greater than about 99%.

Cannabinoid acids suitable for use in the present technology are well known, and may include acid group containing cannabinoids acid forms of cannabinoids or acid group containing cannabinoid, cannabinoid precursors, non-decarboxylated cannabinoids, or derivatives thereof. Suitable cannabinoic acids include, without limitation, tetrahydrocannabinolic acid (THCA), cannabidiolic acid (CBDA), cannabinolic acid (CBNA), cannabichromenic acid (CBCA), cannabigerolic acid (CBGA), cannabinodiolic acid (CBNDA), cannabicyclolic acid (CBLA), cannabivarinic acid (CBVA), tetrahydrocannabivarinic acid (THCVA), cannabidivarinic acid (CBDVA), cannabichrovarinic acid (CBCVA), cannabigerovarinic acid (CBGVA), cannabigerol monomethylether (CBGM), cannabigerolic acid monomethylether (CBGAM), and the like or derivatives thereof, or a combination of any two or more thereof.

The amount of cannabinoic acid in the composition can be determined based on the end use or application of the composition. For example, for use in vaporizer devices, the compositions may include cannabinoic acid in an amount of about 1 to about 30 percent by weight, of the total weight of the composition. In case of other applications where higher concentrations of cannabinoic acid are desired, the compositions may include cannabinoic acid in an amount of about 20 to about 50 percent by weight, of the total weight of the composition.

In one aspect, provided herein are stable compositions that may include one or more active cannabinoids, one or more cannabinoic acid and a carrier (solvent). The carrier may include a single solvent or a blend of multiple solvents, and can be a solvent material in which the desired cannabinoic acid has moderate-to-high solubility, which can be easily removed, and which imparts minimal flavor or aroma to the cannabinoid product. The carrier may include water, alcohols (e.g., C₁-C₄ alcohols), hydrocarbons, ethers, carrier oils, thinning agents, terpenes, or saturated fats. Suitable carriers include, without limitation, methanol, ethanol, isopropanol, butanol, propane, butane, pentane, hexane, heptane, petroleum ether, methyl tertbutyl ether, diethyl ether, carbon dioxide, olive oil, vegetable oil, α-bisabolol, lauric acid, myristic acid, palmitic acid, stearic acid, terpenes and the like or a combination of two or more thereof.

In one aspect, provided herein are stable compositions that may include one or more active cannabinoids, one or more cannabinoic acid, and a flavor or fragrance modifier. Suitable flavor or fragrance modifier may include one or more terpenes or a terpene blend. The terpene blends may be prepared or commercially sourced. In some embodiments, the terpenes can be used as both a solvent and a flavor/fragrance modifier. Suitable terpenes are known in the art, and may include those in which the cannabinoic acid and/or the active cannabinoid has a good solubility. Illustrative terpenes may include, without limitation, α-bisabolol, bergotamene, camphene, camphor, 3-carene, β-caryophyllene, cedrene, citronellol, cymene, eucalyptol, farnesene, fenchol, fenchone, geraniol, guaiol, humulene, isopropyltoluene, isopulegol, isoborneol, linalool, δ-limonene, 1-menthol, β-myrcene, nerol, nerolidol, α- or β-pinene, α- or β-phellandrene, ocimene, sabinene hydrate, α-terpinene, γ-terpinene, terpineol, terpinolene, thujene, valencene, ylangene, and the like, or a combination of any two or more thereof. Many other terpenes are known in the art and commercially available, and can be used in the compositions.

In an embodiment of the present technology, the cannabinoid composition may include about 1 to 20 wt. % cannabinoic acid about 1 to 20 wt. % carrier (e.g., flavor or fragrance modifier), and about 50 to 98 wt. % active cannabinoid. In certain embodiments, the composition may include about 1 to 6 wt. % flavor or fragrance modifier, about 3-10 wt. % cannabinoic acid, the remainder being active cannabinoid.

Cannabinoid compositions according to the present technology can be formulated in various concentrations and forms. For example, a cannabinoid composition can exist as a dilute material that may include an active cannabinoid, a cannabinoic acid, and a carrier; as a partially dilute material wherein the carrier is partially removed; as a neat material with no carrier; or as a material for incorporation into various other forms (e.g., active cannabinoid, a cannabinoic acid, and neutraceutically or pharmaceutically acceptable excipients). Consequently, the concentration of active cannabinoid and cannabinoic acid will clearly differ based on the amount of other constituents present in the composition. The concentration of active cannabinoid and cannabinoic acid will also depend on the particular end use product.

The present technology also provides novel methods for preparing the cannabinoid compositions described herein. Accordingly , in one aspect, provided herein are a novel and cost-effective methods for preventing easy and rapid oxidation of active cannabinoids when exposed to air into to their quinone analogues and other byproducts with cannabis-derived ingredients. For example, provided are two strategies to minimize this reaction from proceeding that rely on latent cannabinoic acid. First is to strategically target a final product that contains less than 80%, for example about 4%-20% by mass of the starting cannabinoic acid during the decarboxylation step. The second method entails full conversion of the cannabinoic acid into the active analogue followed by addition of a cannabinoic acid at about 1-80%, about 1-60%, about 1-40% or about 1-20% by mass, depending on the cannabinoic acid added. The small latent residual acidity significantly prevents the oxidation of the oil and thus reduces color change even when exposed to air for extended periods of time and/or high temperatures (about 70° C.)—both catalysts for oxidation of these compounds. In some embodiments, having over about 20% of the cannabinoic acid may negatively affect the viscosity of the solution, resulting in a glass-like solid that is less conducive for use in certain applications such as in vaping devices. However, it is possible to include higher amounts of the cannabinoic acid for certain applications as demonstrated by the methods of the present technology. As a proof of concept, it was shown that about 5% THCA in a solution of THC relative to a control with 0% THCA has significantly less oxidation when produced either by controlling the reaction endpoint or by exogenous addition of THCA to the fully melted THC solution. The results provide a simple method for preventing oxidation of many high purity cannabinoid extracts without using any non-cannabis derived compounds that may pose a health risk (e.g., inorganic and organic acids).

In one aspect, provided herein are methods for making a stabilized cannabinoid composition, wherein the method includes combining an active cannabinoid, a cannabinoic acid, and, optionally, a carrier to provide a product; wherein the acid is present in an amount effective to improve the stability of the composition. The active cannabinoid may be combined with the cannabinoic acid in various ways. In certain embodiments, the method may include adding the solid cannabinoic acid to the active cannabinoid to provide a mixture, heating the mixture to provide the cannabinoid composition and cooling the cannabinoid composition. In certain embodiments, the method may include solvating the cannabinoic acid into the active cannabinoid, without converting it to the active cannabinoid. In certain embodiments, the method may include heating the cannabinoic acid to a temperature sufficient to provide a glass-like material and adding the glass-like material in to the active cannabinoid. In certain embodiments, the method may include solubilizing the cannabinoic acid in a carrier to provide a solution and adding the solution to the active cannabinoid. In certain embodiments, the method may include adding a flavor modifier, a fragrance modifier, a taste modifier or other perceptible aromatic modifiers to the composition. In certain embodiments, the method may include adding a flavor modulating amount of at least one flavor modifier, a fragrance modulating amount of at least one fragrance modifier, a taste modulating amount of at least one taste modifier, or a combination of any two or more thereof. Suitable modifiers may include chemicals such as terpenes, thiols, esters, ketones, and aldehydes. The present technology contemplates using such other chemicals in alone or in conjunction with terpenes.

In one aspect, provided herein are methods for incorporating acidic cannabinoids into active cannabinoids. These methods can be largely categorized into two classes: (1) carrier-containing methods, and (2) carrier-free methods. In the carrier-containing methods, the cannabinoic acid is dissolved into a liquid solvent with high solubility, following which the carrier solution is injected into the active oil, heated, and homogenized. In the carrier-free methods, solvents are not required. Rather the solid cannabinoic acid is either melted or directly added into active cannabinoid oil in a controlled fashion to target a desired cannabinoic acid concentration. To obtain a higher cannabinoic acid to active cannabinoid ratio, the solid cannabinoic acid can be first melted resulting in a glass like material that is flowable at moderate temperatures. The glass like material is then heated and combined with the active cannabinoid melted oil and homogenized to obtain the desired ratio. The wide array of methods provided here ensures that the cannabinoic acid can effectively be introduced into the product during various times of production.

In one aspect, provided are carrier-containing methods for incorporating acidic cannabinoids into active cannabinoids. In an embodiment of the present technology, the method may include dissolving one or more acidic cannabinoids into a carrier and contacting the solution with active cannabinoid. The carrier can be any low boiling point solvent in which the desired cannabinoic acid has moderate-to-high solubility, which can be easily removed and which imparts minimal flavor or aroma to the cannabinoid product. The solvent can be removed using conventional methods such as applying vacuum and/or mild heat.

Suitable solvents may include alcohols, hydrocarbons, carrier oils, thinning agents, sesquiterpene, diterpene, or saturated fats. Illustrative solvents may include, without limitation, ethanol, methanol, isopropanol, butanol, pentane, hexane, heptane, vegetable oil, α-bisabolol, lauric acid, myristic acid, palmitic acid, stearic acid, and the like or a combination of two or more thereof.

In an embodiment of the present technology, the carrier-containing method may include dissolving one or more acidic cannabinoids into a one or more terpenes and contacting the solution with active cannabinoid. In some embodiments, the terpenes can be used as both a solvent and a flavor/fragrance modifier, in which case removal of the solvent is not required.

In any embodiment, the carrier constitutes at least about 1 by weight of the total composition, at least about 5, at least about 10%, at least about 15%, at least about 20%, at least about 30%, at least about 40%, at least about 50%, at least about 60% or at least about 90% by weight. In certain embodiments, the solvent constitutes up to about 99.99% by weight of the total composition, up to about 95%, up to about 90%, up to about 85%, up to about 80%, up to about 70% or up to about 60% by weight of the total weight of the composition. In certain embodiments, the solvent constitutes about 1% to about 90% of the total weight of the composition, including without limitation, about 10% to about 80%, about 20% to about 75%, about 30% to about 80%, about 40% to about 75%, about 50% to about 70%, or about 55% to about 65% of the total weight of the composition, or any range including and/or in-between any two of these values.

In another aspect, provided are carrier-free methods for incorporating acidic cannabinoids into active cannabinoid. In an embodiment of the present technology, the carrier-free method may include melting a pure active cannabinoid isolate under suitable conditions to achieve the desired level of cannabinoic acid concentration. The method may include, for example, melting the pure active cannabinoid isolate under inert conditions at a suitable temperature and for a suitable period. The temperature and time can be modulated depending on the type of active cannabinoid and the desired concentration of acidic cannabinoid. For example, high purity cannabinoic acid (e.g., THCA) is isolated and then melted into its active cannabinoic analog to retain a fraction of the cannabinoic acid (e.g., THC) in the melt. As the cannabinoic acid melts, the concentration can be monitored via suitable analytical methods (e.g., HPLC). When the appropriate concentration of the Cannabinoid acid is reached in the active cannabinoid, the reaction temperature is quenched to prevent further decarboxylation.

In an embodiment of the present technology, the carrier-free method may include contacting the cannabinoic acid with an active cannabinoid. The solid cannabinoic acid can be added as such or can be ground into a microcrystalline powder to help facilitate solubilization. If the solid has poor solubility and dissolves slowly into the oil, even at higher temperatures, excess cannabinoic acid can be added to achieve the desired concentration. The method can be conducted in the absence of a solvent. The method may further include removing any residual solid cannabinoic acid using suitable separation techniques, e.g., filtration.

In an embodiment of the present technology, the carrier-free method may include melting the cannabinoic acid and contacting it with an active cannabinoid. The method may include preparing a high concentration cannabinoic acid glass (e.g., >20% by weight), and combining it with an active cannabinoic oil. The high cannabinoic acid concentration minimizes the amount of the glass to be added to active cannabinoid oil necessary to achieve a desired cannabinoic acid concentration in the final product. The cannabinoic acid glass can be formed by first melting the pure cannabinoic acid, optionally under inert conditions to convert it to a liquid. The liquid cannabinoic acid can be further heated for a suitable period of time to convert it to glass-like material, which can then be contacted and homogenized with the active cannabinoid.

The heating conditions for the cannabinoic acid or the composition, including time and temperature will vary depending on the particular cannabinoic acid and/or active cannabinoid being used. For example, in some embodiments, the cannabinoic acid is heated to a temperature and for a time period where it melts. In other embodiments, the cannabinoic acid is heated to a temperature and for a time period where it provides a glass-like material. In certain embodiments, the cannabinoic acid or the composition may be heated to a temperature between 40° C. and 250° C., between 45° C. and 200° C., between 50° C. and 150° C., between 55° C. and 140° C., between 60° C. and 130° C., between 65° C. and 125° C., between 70° C. and 120° C., between 75° C. and 115° C., between 80° C. and 110° C., between 85° C. and 105° C., or between 90° C. and 100° C., or any range including and/or in-between any two of these values. In certain embodiments, the cannabinoic acid or the composition may be heated for about 5 min to about 24 h, about 10 min to about 15 h, about 20 min to about 10 h, about 30 min to about 8 h, about 45 min to about 6 h, about 60 min to about 5 h, about 2 h to about 4 h or about 2.5 h to about 3.5 h, or any range including and/or in-between any two of these values.

In one aspect, provided is a method for preparing a stabilized cannabinoid composition, the process may include providing an active cannabinoid at a purity of greater than 90 wt. %, and adding a cannabinoic acid to the active cannabinoid. The cannabinoic acid can be added in an amount of about 1 to about 50 wt. %, about 2 to about 30 wt. %, or about 3 to about 10 wt. %.

In another aspect, provided is a method for preparing a stabilized cannabinoid composition, the process may include thermally decarboxylating a cannabinoic acid at a purity of greater than 90 wt. % to an active cannabinoid, and terminating the decarboxylation when the composition contains from about 1 to about 50 wt. %, about 2 to about 30 wt. %, or about 3 to about 10 wt. % of the cannabinoic acid.

In some embodiments, the active cannabinoid used in the compositions and methods may include Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), or cannabidiol, or a combination of any two or more thereof. In some embodiments, the cannabinoic acid used in the compositions and methods is an endogenous cannabinoic acid. In some embodiments, the cannabinoic acid used in the compositions and methods may be 2-Δ⁸-tetrahydrocannabinolic acid (2-Δ⁸-THCA), 4-Δ⁸-tetrahydrocannabinolic acid (4-Δ⁸-THCA), 9-Δ⁸-tetrahydrocannabinolic acid (9-Δ⁸-THCA), 2-Δ⁹-tetrahydrocannabinolic acid (2-Δ⁹-THCA), 4-Δ⁹-tetrahydrocannabinolic acid (4-Δ⁹-THCA), 9-Δ⁹-tetrahydrocannabinolic acid (9-Δ⁹-THCA), 2-Δ¹⁰-tetrahydrocannabinolic acid (2-Δ¹⁰-THCA), 4-Δ¹⁰-tetrahydrocannabinolic acid (4-Δ¹⁰-THCA), 9-Δ¹⁰-tetrahydrocannabinolic acid (9-Δ¹⁰-THCA), cannabidiolic acid, or cannabigerolic acid, or a combination of any two or more thereof. In some embodiments, the cannabinoic acid is a mixture of two or more of 2-Δ⁸-tetrahydrocannabinolic acid (2-Δ⁸-THCA), 4-Δ⁸-tetrahydrocannabinolic acid (4-Δ⁸-THCA), 9-Δ⁸-tetrahydrocannabinolic acid (9-Δ⁸-THCA), 2-Δ⁹-tetrahydrocannabinolic acid (2-Δ⁹-THCA), 4-Δ⁹-tetrahydrocannabinolic acid (4-Δ⁹-THCA), 9-Δ⁹-tetrahydrocannabinolic acid (9-Δ⁹-THCA), 2-Δ¹⁰-tetrahydrocannabinolic acid (2-Δ¹⁰-THCA), 4-Δ¹⁰-tetrahydrocannabinolic acid (4-Δ¹⁰-THCA), 9-Δ¹⁰-tetrahydrocannabinolic acid (9-Δ¹⁰-THCA), cannabidiolic acid, or cannabigerolic acid, or a combination of any two or more thereof. In some embodiments, the active cannabinoid is Δ⁹-THC, and the endogenous cannabinoic acid is 2-Δ⁹-THCA or 4-Δ⁹-THCA.

In one aspect, the methods described herein may further include adding one or more terpenes to the stabilized cannabinoid composition. Illustrative terpenes include, but are not limited to, myrcene, β-caryophyllene, α or β-Pinene, α or β-phellandrene, limonene, terpinolene, linalool, pinene, terpineol, fenchyl alcohol, α-bisabolol, camphene, terpinolene, humulene, geraniol, camphor, cedrene, 1-menthol, cis-β-ocimene, trans-β-ocimene, terpinene, delta-3-carene, isoborneol, nerol, valencene, farnesene (t), fenchone, ocimene, bergotamene, thujene, ylangene, sabinene hydrate, and the like, or a combination of any two or more thereof. Many other terpenes are known in the art and commercially available, and can be used in the compositions.

In another aspect, provided is a stabilized cannabinoid composition that may include about 1 to about 50 wt. %, about 2 to about 30 wt. %, or about 3 to about 10 wt. % of cannabinoic acid, and a high purity active cannabinoid.

In one aspect, provided is a method of purifying tetrahydrocannabinolic acid (THCA), wherein the method includes providing a crude THCA isolate extracted from cannabis, and centrifuging the crude THCA isolate to provide crystals of THCA at a purity greater that those present in the crude THCA isolate.

For various compositions and methods described herein, depending on the cannabinoid product, the active cannabinoid may be present in the cannabinoid formulation in an amount of about 1 wt. % to 99.9 wt. %, including, without limitation, about 5 to about 98 wt. %, about 10 to about 90 wt. %, about 20 to about 80 wt. %, about 30 to about 70 wt. %, about 40 to about 60 wt. %, or about 50 to about 55 wt. %, and ranges in between and including the two values. In any embodiment, the active cannabinoid may be present in an amount of about 50 to about 99.9 wt. %, including, without limitation, about 50 to about 99 wt. %, about 55 to about 95 wt. %, about 60 to about 90 wt. %, about 65 to about 85 wt. %, or about 70 to about 80 wt. %, and ranges in between and including the two values. In any embodiment, the active cannabinoid may be present in an amount of about 85 to about 99.9 wt. %, including, without limitation, about 90 to about 99 wt. %, about 90 to about 98 wt. %, about 90 to about 97 wt. %, about 90 to about 96 wt. %, about 90 to about 95 wt. %, or about 90 to about 94 wt. %, and ranges in between and including the two values. In any embodiment, the active cannabinoid may be present in an amount of about 40 to about 99 wt. %. The active cannabinoid may include a combination of two or more active cannabinoids.

For various compositions and methods described herein, depending on the cannabinoid product, the cannabinoic acid or latent cannabinoic acid may be present in the cannabinoid composition in an amount of about 1 wt. % to 99.9 wt. %, including, without limitation, about 2 to about 90 wt. %, about 5 to about 80 wt. %, about 10 to about 70 wt. %, about 15 to about 60 wt. %, about 20 to about 50 wt. %, about 25 to about 45 wt. %, or about 30 to about 40 wt. %, and ranges in between and including the two values. In any embodiment, the cannabinoic acid may be present in an amount of about 1 to about 60 wt. %, including, without limitation, about 1 to about 50 wt. %, about 5 to about 45 wt. %, about 10 to about 40 wt. %, about 15 to about 35 wt. %, about 20 to about 40 wt. %, or about 30 to about 35 wt. %, and ranges in between and including the two values. In any embodiment, the cannabinoic acid may be present in an amount of about 0.1 to about 20 wt. %, including, without limitation, about 1 to about 12 wt. %, about 2 to about 11 wt. %, about 3 to about 10 wt. %, about 4 to about 10 wt. %, about 5 to about 10 wt. %, or about 6 to about 10 wt. %, and ranges in between and including the two values. In any embodiment, the cannabinoic acid may be present in an amount of about 1 to about 60 wt. %. The cannabinoic acid may include a combination of two or more cannabinoic acids.

For various compositions and methods described herein, the ratio of active cannabinoid to the cannabinoic acid may range from about 100:1 to about 1:10, including without limitation, about 25:1 to about 1:1, about 10:1 to about 1:1, about 8:1 to about 1:1, about 7:1 to about 1:2, about 6:1 to about 1:3, about 5:1 to about 1:4, about 4:1 to about 1:5, about 3:1 to about 1:6, about 2:1 to about 1:7, or about 1:1 to about 1:8, and ratios in between and including the two values. In any embodiment, the ratio of active cannabinoid to the cannabinoic acid may range from about 5:1 to about 1:2. In any embodiment, the ratio of active cannabinoid to the cannabinoic acid may range from about 2:1 to about 1:2. In any embodiment, the ratio of active cannabinoid to the cannabinoic acid may range from about 10:1 to about 1:1. In any embodiment, the ratio of active cannabinoid to the cannabinoic acid may be about 1:1.

For various compositions and methods described herein, depending on the cannabinoid product, the endogenous terpenes may be present in the cannabinoid composition in an amount of less than about 30 wt. %, including less than about 25 wt. %, less than about 20 wt. %, less than about 10 wt. %, less than about 8 wt. %, less than about 5 wt. %, less than about 4 wt. %, less than about 3 wt. %, or less than about 2 wt. %, and values in between. In any embodiment, the endogenous terpenes may be present in an amount of less than about 3 wt. %, less than about 2 wt. %, or less than about 1 wt. % of the total weight of the composition. In any embodiment, the endogenous terpenes may be present in the composition in an amount of about 0 to about 10 wt. %, including, without limitation, about 0.5 to about 8 wt. %, about 1 to about 7 wt. %, about 1.5 to about 5 wt. %, or about 0 to about 2 wt. %, and ranges in between and including the two values.

In one aspect, the present technology relates to composition comprising about 1 wt. % to about 60 wt. % of a cannabinoic acid, about 40 wt. % to about 99 wt. % of active cannabinoid, and less than 3 wt. % of endogenous terpenes. In another aspect, the present technology relates to composition comprising about 1 wt. % to about 60 wt. % of a cannabinoic acid, about 40 wt. % to about 99 wt. % of active cannabinoid, and less than 2 wt. % of endogenous terpenes.

The stabilized cannabinoid composition described herein can be used in products such as edible products, aerosol products, fragrance products, flavor products, inhalable products, consumer products, personal care products, and household products. In certain embodiments, the edible product is a food product or a beverage product. In certain embodiments, the stabilized cannabinoid composition described herein can be used in vapes, foods, beverages, medicinal products, herbal products, health products, nutritional products, and the like. They can be delivered using modes of delivery, such as smoking, using a vapourizer, eating, or drinking.

In another aspect, the present technology relates to various products that may include the stabilized cannabinoid compositions described herein. Illustrative products include, but are not limited to edible products, aerosol products, fragrance products, flavor products, or inhalable products. In certain embodiments, an edible product comprising a composition described herein is provided. In certain embodiments, the edible product is a food or beverage product. In certain embodiments, the food product may include candy, licorice, taffy, chews, gummies, jelly bean, and the like. In certain embodiments, the beverage is beer, any alcohol containing beverage, or other non-alcohol containing beverage product. In certain embodiments, a flavor and fragrance product comprising a composition described herein is provided. In certain embodiments, an inhalation product comprising the composition described herein is provided. In certain embodiments, the inhalation product is a vaping composition. In certain embodiments, a flavor and/or fragrance delivery system comprising the composition described herein is provided.

Depending upon the end application, the compositions may include other ingredients, such as additives, surfactants, co-solvents, propellants, other flavoring agents, medicinal agents, perfumes, stabilizers, thickeners, binders, preservatives, emulsifiers, essential oils, water, salt, sweeteners, gelatin, food additives, colorants, excipients, diluents, fatty acids, triglycerides, terpenes, flavanoids and the like or a combination of any two or more thereof. For example, the compositions may include other phyto-derived compounds, i.e., nitrogenous compounds, amino acids, proteins, enzymes, glycoproteins, hydrocarbons, alcohols, aldehydes, ketones, fatty acids, esters and lactones, steroids, terpenes, non- cannabinoid phenols, flavonoids, vitamins and pigments. In certain embodiments, the compositions may include commonly used terpenes, sesquiterpenes, and their oxygenated derivatives, or canninoid products. In certain embodiments, the compositions may include flavor modifiers, fragrance modifiers or a combination thereof. In certain embodiments, the flavor modifiers may include terpene compounds. Illustrative terpene compounds are described herein. In certain embodiments, the terpenes may be exogenously added to the composition or endogenously present in the compositions. In certain embodiments, the terpenes may be exogenously added to the composition.

In another aspect, the present technology relates to a composition comprising a cannabinoic acid, an active cannabinoid and one or more terpenes. In certain embodiments, the terpenes may be exogenously added to the composition. In certain embodiments, the terpenes are not endogenous to the composition. In certain embodiments, the terpenes may be a mixture of endogenous and exogenously added terpenes. The amounts of cannabinoic acid and active cannabinoid are described hereinabove. In certain embodiments, the one or more terpenes constitute at least about 30% by weight of the total composition, at least about 4%, at least about 5%, at least about 8%, at least about 10%, at least about 12% or at least about 15% by weight. In certain embodiments, the terpenes constitute up to about 90% by weight of the total composition, up to about 75%, up to about 50%, up to about 30%, up to about 20%, up to about 15% or up to about 100% by weight of the total weight of the composition. In certain embodiments, the terpenes constitutes about 2% to about 50% of the total weight of the composition, including without limitation, about 3% to about 20%, about 4% to about 15%, or about 5% to about 10%, of the total weight of the composition, or any range including and/or in-between any two of these values. In certain embodiments, the composition comprises about 4 wt. % to about 10 wt. % of a cannabinoic acid, about 85 wt. % to about 93 wt. % of active cannabinoid and about 4 wt. % to about 15 wt. % of one or more terpenes. In certain embodiments, the one or more terpenes are exogenously added to the composition.

The present invention, thus generally described, will be understood more readily by reference to the following examples, which are provided by way of illustration and are not intended to be limiting of the present invention.

### EXAMPLES

### Example 1: High Purity THCA Isolation

High purity (e.g., >94% or >98%) THCA solid was produced by extraction of cannabis using butane at −40° C. for 10 min. The resulting liquid was allowed to warm to room temperature over the course of 2 days, resulting in large-grain crystallites to form in the solution. The crystallites, identified as THCA, were mechanically separated and further purified via centrifugation resulting in high purity microcrystalline THCA to be easily separated from the remaining larger crystallite solid. This solid was confirmed to be over 94% or in some cases 98% THCA, with small amounts of other cannabinoids present.

### Example 2: UV-Visible Spectroscopy

The color purities of THC oils with and without acidic cannabinoids were measured using a LabTech BlueStar A UV-Vis Spectrophotometer. The data collection range was from 250 nm to 1100 nm at a scan rate of 3 nm per second. About 1 g of sample was added to scintillation vial followed by addition of 5 mL of ethanol. The solution was agitated for 2 min in order to fully dissolve the oil and transferred to a UV-Vis quartz cuvette. An ethanol background was collected prior to running the samples to reduce solvent.

### Example 3: Controlled THCA Conversion to THC

1 Kg of solid, high purity THCA (>98%) was divided into two vessels, one acting as a control (C1) and the other as the experimental sample (E1). A programmable vacuum oven with multiple zones was then set to heat the control and experimental sample at different rates: The “hot” zone for the control was ramped to about 240° F. and dwelled for 15 hours while the “cold” zone was ramped to about 200° F. and dwelled for 15 hours. Both zones were evacuated followed by addition of an N₂ atmosphere to minimize oxidation during the reaction. The zones were then slow cooled to room temperature and exposed to air. At this stage, C1 was a pure THC with no remaining THCA, while E1 contained about 5.9 wt. % THCA.

As necessary, about 5 g of a non-acidic cannabinoid oil was added into a scintillation vial. Approximately 0.26 g of the desired acidic cannabinoid was then added to the oil (about 5% nominal amount by weight). The mixture was then gently heated to homogenize the resulting sample. After mixing, if any solid precipitate was present, the oil was filtered.

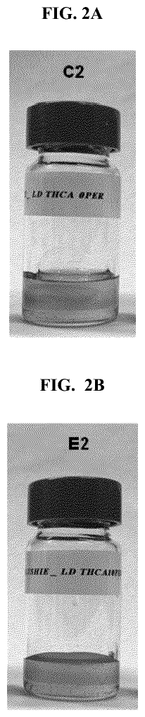

After only about 30 min of air exposure, a red film had formed in C1 (FIG. 1A), whereas E1 had minimal color change (FIG. 1B). This was confirmed by UV-Visible spectra for C1, E1, and CBDA-E1, as described below, wherein greater visible absorption was observed in C1 than E1 or CBDA-E1, correlating with the darker physical color. After confirming that latent THCA may significantly reduce the oxidation and discoloration of THC, sample C1 was then split into two separate samples, C2 and E2. The samples were heated to about 70° C. to decrease the viscosity of the solutions to a thin liquid. At this point, the solutions of C2 (FIG. 2A) and E2 (FIG. 2B) were observed to have a reduced red color, indicative of THCQ and other byproducts dissolving into the bulk of the solution. No additives have been added to either vial, and similar color is observed for each sample, indicating that they are homogenous.

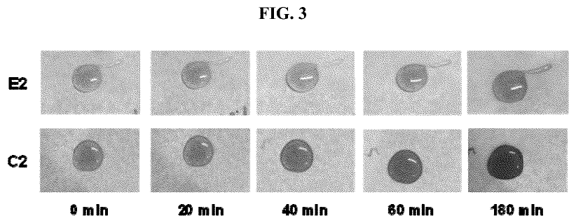

About 10 wt. % solid THCA crystallites were then added into E2 with stirring, while C2 was stirred with no addition of THCA. The solid THCA crystallites did not completely dissolve, and subsequent analysis revealed that the THCA concentration present in the solution was 6 wt. %. To quickly screen and easily monitor the color change in air, about 5 mL of each were deposited onto a polytetrafluoroethylene (PTFE) substrate and allowed to cool. Minimal color change was observed in E2, whereas C2 exhibited has significant color change after only about 2 hours (FIG. 3). After 3 h, the dark red color previously seen was not evident due to homogenization of THCQ and other byproducts into the solution.

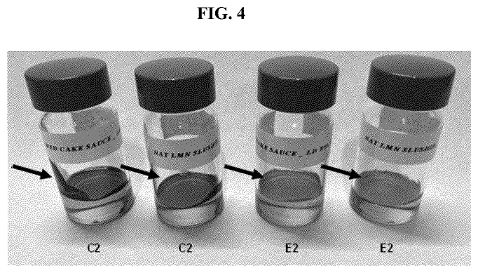

About 5 g of E2 and 5 g of C2 was then transferred into four separate vials (2 for each sample) and cooled. Similar trends were then observed in oxidation between these samples as in FIG. 3. The most evident change in color was observed after the vials were allowed to cool for about 24 hours as shown in FIG. 4. Significant darkening at the surface of C2 is present while minimal color change occurred in E2. These results show that ambient conditions can negatively affect high purity THC due to oxidation, but it can be prevented by addition of a cannabinoic acid such as THCA.

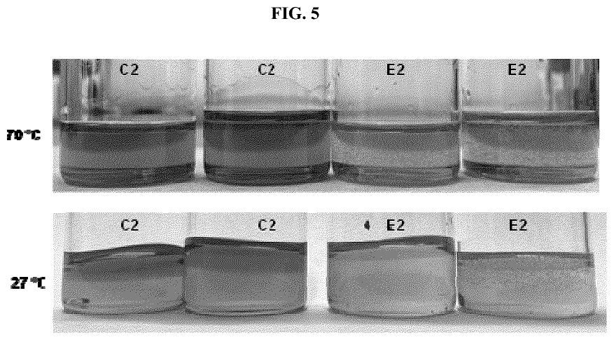

To study if other additives can prevent or mask discoloration such as that seen in FIG. 4, 7 wt. % of botanical terpenes was added to each sample of C2 and E2. After vigorous stirring, each sample appeared homogenous. Nonetheless, there is a significant darkening of color for samples that showed prior oxidation (FIG. 5, 70° C.). However, cooling the samples and after 12 hours, the homogenous solution separated (FIG. 5, 27° C.). The minimal color change in both E2 samples further confirms that the presence of acidic cannabinoids may help prevent, or minimize, oxidation.

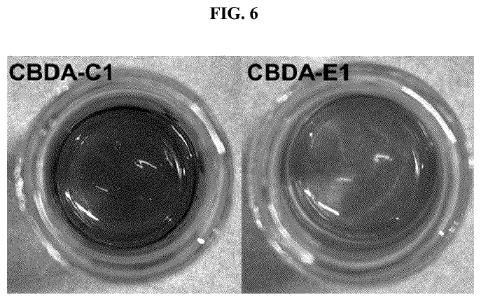

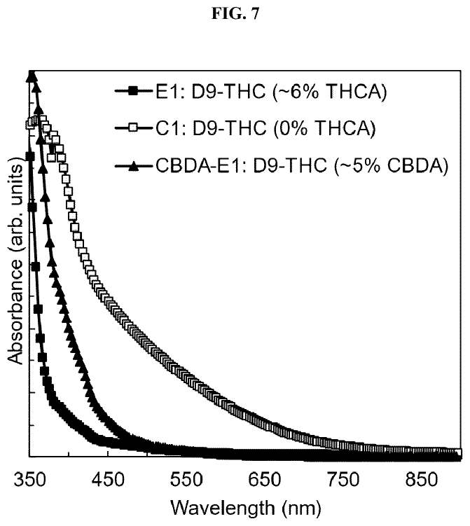

The effect of CBDA on reduction in color changes was tested by adding a nominal 5% CBDA to THC containing no latent acidic cannabinoids. As shown in FIG. 6, it was observed that there is a significant color improvement in the sample with CBDA added (CBDA-E1), compared to that with no CBDA added (CBDA-C1). The spectra of CBDA-E1 is shown in FIG. 7. These results show that this method of minimizing oxidation and color changes include using other acidic cannabinoids beyond THCA.

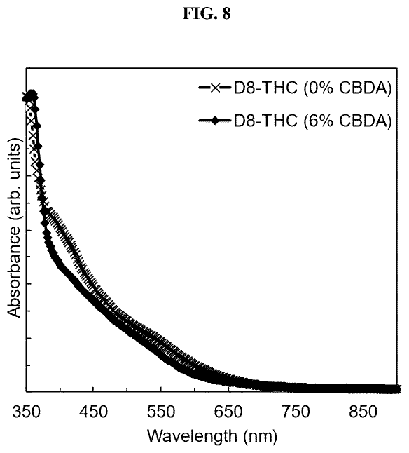

Similar experiments were conducted with both D8-THC and D9-THC as the non-acidic cannabinoid and CBDA as the acidic cannabinoid. About 5% CBDA by weight was added to a sample of D9-THC and homogenized under mild heat (about 70° C.), resulting in a yellow solution with minimal discoloration. A final sample of D8-THC then had 5% by weight CBDA added to determine if it likewise has a beneficial activity (FIG. 8). It was observed that while the sample with 5% CBDA added has less visible absorption, it is less apparent than that of D9-THC. The overall discoloration of the starting D8-THC sample was not as severe as the D9-THC sample, which may explain why the overall improvement was not as significant. Nonetheless, the reduction in visible absorption indicates that the acidic cannabinoid is still capable of alleviating color changes and may be more drastic if the D8-THC oil color is more severely oxidized and discolored.

### Example 4: Beam Test

Beam test is a well-known experiment used to detect THC. Using the beam test, the effect of THCA on color changes was studied under more extreme conditions extreme pH changes for a commercially available THC distilled oil sample (0% THCA) (Comparative Sample A) and the THC oil of Example 3 (about 5% THCA) (Sample B) were used to conduct the beam test. A 5% KOH solution in ethanol was prepared. 1 g of the respective oil was added to a scintillation vial followed by addition of 5 mL of the 5% KOH ethanolic solution. The resulting solutions were homogenized and allowed to sit for about 10 min before UV-Vis spectroscopy data collection was commenced. 2 mL of the solution were added to 3 mL of ethanol to dilute the resulting products into appropriate concentration range for the analytical instrument (UV-Vis Spectrophotometer).

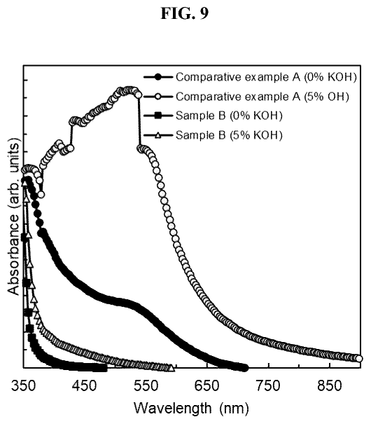

The results of the beam-test are as shown in FIG. 9. Prior to addition of the basic solutions (0% KOH), Sample B had significantly less discoloration than Sample A. This change in visible discoloration was significantly more obvious when 5% KOH was added. While Sample A became a dark violet colored solution with KOH addition, Sample B had a minimal increase in color. These results reinforce the observation that addition of cannabinoic acids (e.g., THCA), cannot only prevent oxidation and discoloration of non-acidic cannabinoids under mild conditions, such as those during commercial cannabis product manufacturing and usage, but can also help prevent discoloration under extreme conditions. Taken together, the data shows that acidic cannabinoids can reduce oxidation and discoloration of non-acidic cannabinoid oils without requiring additives that are not cannabis or hemp derived.

### Example 5: Incorporation of Cannabinoic Acid in to Active Cannabinoid

1 g of high purity (>94%) THCA was added to a scintillation vial. 100 mL aliquots of ethanol or Watermelon Zkittlez botanical terpene blend (Abstrax Tech brand) were then added sequentially until the solid was fully dissolved.

Cannabinoid concentrations were measured by Bel Costa, a 3^{rd} party ISO-certified cannabis-testing lab located in Long Beach, CA. The concentrations were obtained using a high-performance liquid chromatography with a diode-array detector (HPLC-DAD) with calibration curves of each analyte of interest.

Various methods were used to incorporate cannabinoic acid into the active cannabinoid media, and are summarized in Table 1 below.

TABLE 1
Methods of incorporating cannabinoic acid into active cannabinoic media

| Method | | | |
|---|---|---|---|
| # | Method | Classification | Brief Description |
| M1 | Controlled melt of | Carrier-free | Solid cannabinoic acid is melted into active |
| | cannabinoic acid in | | cannabinoic oil in a controlled fashion to |
| | to active | | target a desired cannabinoic acid |
| | cannabinoic | | concentration. |
| M2 | Direct addition of | Carrier-free | Solid cannabinoic acid is added directly to |
| | cannabinoic acid to | | active cannabinoic oil in the desired |
| | active cannabinoic | | concentration. |
| M3 | High concentration | Carrier-free | Solid cannabinoic acid is melted to obtain a |
| | cannabinoic acid:active | | high cannabinoic acid:active cannabinoid |
| | cannabinoid glass | | ratio, resulting in a glass that is flowable at |
| | | | moderate temperatures. Glass is then heated |
| | | | and combined with active cannabinoic oil and |
| | | | homogenized in appropriate ratio. |
| M4 | Solvent-infused | Carrier- | Solid cannabinoic acid is dissolved into a low |
| | injection | containing | boiling-point solvent that is intended to |
| | | | provide minimal scent or flavor. Infused |
| | | | solvent is homogenized into active |
| | | | cannabinoic oil in desired concentration, |
| | | | followed by evaporation of solvent. |
| M5 | Flavor-infused | Carrier- | Solid cannabinoic acid is dissolved into a |
| | injection | containing | flavor-blend that is intended to be used in a |
| | | | final formulation of the active cannabinoic |
| | | | product. Infused flavor formulation is |
| | | | homogenized into the active cannabinoic oil. |
| M6 | Carrier-free Wipe- | Carrier-free | Solid cannabinoic acid is melted to obtain a |
| | film distillation | | desired cannabinoic acid:active cannabinoid |
| | | | ratio using wiped film distillation. |
| M7 | carrier-containing | Carrier- | Solid cannabinoic acid is dissolved into a low |
| | Wipe-film | containing | boiling point solvent. The resulting solution is |
| | distillation | | then distilled using wiped-film distillation to |
| | | | result in a desired cannabinoic acid:active |
| | | | cannabinoid ratio. |

### Example 6: Carrier-Free Methods

Method M1: Controlled Melt of Cannabinoic Acid Into Active Cannabinoid

500 g of >97% THCA was added to a mesh filter and positioned on top of a 64-ounce mason jar and placed into a vacuum oven. About 1×10⁻² torr vacuum was then applied for 10 min and the oven was backfilled with nitrogen. The temperature of the oven was then increased to 120° C. and maintained for 8 h to thermally convert THCA into THC followed by slow cooling to room temperature. A 2 g sample of the resulting extract was then capped and tested for THCA and THC concentration, which are summarized in Table 2. Final yield was 435 g (87% yield).

Method M2: Direct Addition of Cannabinoic Acid to Active Cannabinoid

125 g of >97% THCA was ground in to a microcrystalline powder and added to 2081 g THC at a temperature of 60° C. and maintained for 1.5 h with continuous stirring followed by slow cooling to room temperature. The resulting mixture was filtered and collected. A 2 g sample of the resulting extract was then capped and tested for THCA and THC concentration, which are summarized in Table 2. Final yield was 1929 g (89% yield).

Method M3: High Concentration Cannabinoic Acid: Active Cannabinoid Glass

A 100 g sample of 20:70% THCA to THC was formed by heating about 95% pure THCA to a temperature of 130° C. under argon in a 3-neck round bottom flask. For the first 30 min, the solid slowly converted into a liquid. Once no visible solid was present in the resulting oil, the solution was further heated for 1 h. The resulting oil was tested and confirmed to contain approximately 21.1% THCA in the resulting glass (M3A in Table 2). 5 g of the resulting high-THCA glass was heated to 60° C. and poured into a solution of 5 g of THC oil followed by homogenization. The resulting product had about 11.5% THCA (M3B in Table 2).

Method M4: Solvent as a Carrier

A 1.25 g/mL solution of THCA in ethanol was prepared by dissolving 62.5 g THCA in to 50 mL ethanol at 70° C. 48 mL of the prepared THCA ethanolic solution was added to 1 Kg of THC (about 95%), and homogenized at 60° C. The ethanol was then evaporated by heating the solution to 70° C. under vacuum and agitation for 6 h. The resulting THCA content was about 4.1%

Method M5: Flavor Blend as a Carrier

A 500 g solution of THC, THCA, and Watermelon Zkittlez botanical terpene blend (Abstrax Tech brand) (89%, 6%, and 5% of the final product by mass, respectively) was prepared. 30 g of THCA was dissolved into 25 g of the terpene blend by heating the solution at 60° C. for 30 min, in a closed container with agitation. After the crystals had fully dissolved, the THCA-terpene blend solution was poured directly into 445 g of THC. The resulting solution was heated at 60° C. for 15 min and stirred to homogenize the sample. The resulting THCA content was about 4.1%.

Method M6: Carrier-Free Wiped-Film Distillation

187 g of high purity THCA was added to the feed flask of a 2″ Pope Wiped Film Distillation System equipped with a heat jacket. This method involves separating the compounds from highly viscous liquids using a vertical evaporator (referred to as a column). The pope distillation unit was placed under vacuum reaching a pressure of approximate 500 mTorr or lower if possible. The low vacuum pressure assists in melting the THCA to its THC. Three cold/heat circulators were used in this experiment: an internal condenser (75° C.), an external condenser (−15° C.), and a cold trap condenser (−60 ° C.). The column was heated to 170-180° C. Once the desired column temperature and vacuum pressure was reached, the distillation began and the melted THCA was released into the column of the unit where the wipers came into contact with the glass surface. The decarboxylated (liquid) THC acts as a carrier of THCA as the material passes through the column, decarboxylating at a rate, which was found to be ideal for retaining THCA with the above parameters. The resulting THCA/THC solution was then collected in the flask attached on to the internal condenser. 102 g of distilled extract was collected. The resulting extract was then capped and tested for THCA and THC concentration. The resulting product had about 91.41% THC, 3.67% THCA, and 94.63% Total THC.

Method M7: Carrier-Containing Wiped-Film Distillation

In this method, about 200 g of high purity THCA was dissolved into 200 g of ethanol. The solution was then added to a feed flask of a Pope 2″ Wiped Film Distillation System equipped with a heat jacket. The distillation unit was placed under vacuum reaching a pressure of approximate 500 mTorr or lower. The low vacuum pressure assists in melting the THCA to THC. Three cold/heat circulators were used in this experiment: an internal condenser (75° C.), external condenser (−15° C.), and a cold trap condenser (−60° C.). The column was heated to 120° C. Once the desired column temperature and vacuum pressure was reached, the distillation began using the solution of THCA in ethanol. The solution was released into the column of the unit where the wipers encountered the glass surface. The high temperature ensured ethanol evaporated to the cold trap condenser, whereas the remaining THCA decarboxylated in the column. The resulting solvent-free extract was then collected in the flask attached on to the internal condenser (48% yield). The resulting extract was then capped and tested for THCA and THC concentration. The resulting product had about 43.8% THC, 55.4% THCA, and 92.35% total THC.

Active cannabinoid and Cannabinoid acid concentrations in the products obtained from methods M1-M7 are summarized in Table 2 below.

TABLE 2

| Method | THCA (%) | THC (%) | Total THC (%)* |
|---|---|---|---|
| M1 | 8.5113 | 89.431 | 96.896 |
| M2 | 4.63919 | 90.58213 | 94.6507 |
| M3A | 21.10793 | 75.86422 | 94.37587 |
| M3B | 11.58465 | 85.31204 | 95.47178 |
| M4 | 4.13432 | 87.81048 | 91.43628 |
| M5 | 4.07463 | 90.05445 | 93.6279 |
| M6 | 3.67 | 91.41 | 94.63 |
| M7 | 43.8 | 55.4 | 92.35 |

*Total THC is defined as: Total THC = (THCA × 0.877) + THC.

### Example 7: Compositions

Compositions and flavor compounds were prepared, which included the cannabinoic oils containing cannabinoic acids in combination with various terpenes. Illustrative compositions are shown in Table 3 below:

TABLE 3

| | Composition | | |
|---|---|---|---|
| Watermelon Zkittlez vape | I | II | III |
| formulation | (wt. %) | (wt. %) | (wt. %) |
| THC oil | 89% | 85% | 75% |
| THCA | 6% | 10% | 20% |
| Watermelon Zkittlez terpenes | 5% | 5% | 5% |
| (Abstrax Tech brand) | | | |

Although THC/THCA are illustrated in the Examples above, the compositions and methods described herein are not limiting to THCA as an antioxidant and THC as the substrate of interest. Other active cannabinoids-cannabinoic acid combinations can likewise be prepared resulting in stable compositions. Addition of various acidic cannabinoids can help stabilize high purity active cannabinoids, with respect to oxidation, and much more.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A method for making a stabilized cannabinoid composition, the method comprising combining an active cannabinoid, a cannabinoic acid, and, optionally, a carrier to provide a product; wherein the acid is present in an amount effective to improve the stability of the composition.

2. The method of claim 1, wherein the active cannabinoid is tetrahydrocannabinol (THC).

3. The method of claim 1, wherein the cannabinoic acid is an endogenous cannabinoic acid.

4. The method of claim 3, wherein the endogenous cannabinoic acid is tetrahydrocannabinolic acid (THCA), cannabidiolic acid (CBDA), cannabinolic acid (CBNA), cannabichromenic acid (CBCA), cannabigerolic Acid (CBGA), cannabinodiolic acid (CBNDA), cannabicyclolic Acid (CBLA), cannabivarinic acid (CBVA), tetrahydrocannabivarinic acid (THCVA), cannabidivarinic acid (CBDVA), cannabichrovarinic acid (CBCVA), cannabigerovarinic Acid (CBGVA), cannabigerol monomethylether (CBGM), cannabigerolic acid monomethylether (CBGAM), or a combination of any two or more thereof.

5. The method of claim 1, wherein the composition comprises a carrier of methanol, ethanol, isopropanol, butanol, propane, butane, pentane, hexane, heptane, petroleum ether, methyl tertbutyl ether, diethyl ether, carbon dioxide, olive oil, vegetable oil, α-bisabolol, lauric acid, myristic acid, palmitic acid, stearic acid, a terpene, or a combination of two or more thereof.

6. The method of claim 1, wherein the active cannabinoid:cannabinoic acid ratio ranges from about 10:1 to about 1:1 by weight.

7. The method of claim 1, wherein the composition comprises from about 50 wt. % to about 99 wt. % of the active cannabinoid and about 1 wt. % to about 50 wt. % of the cannabinoic acid.

8. The method of claim 1, wherein the active cannabinoid has a purity of greater than 95% and/or wherein the cannabinoic acid has a purity of greater than 95%.

9. The method of claim 1, wherein combining active cannabinoid with the cannabinoic acid comprises adding the solid cannabinoic acid to the active cannabinoid to provide a mixture, heating the mixture to provide the cannabinoid composition and cooling the cannabinoid composition.

10. The method of claim 1, wherein combining active cannabinoid with the cannabinoic acid comprises solvating the cannabinoic acid into the active cannabinoid.

11. The method of claim 1, wherein combining active cannabinoid with the cannabinoic acid comprises heating the cannabinoic acid to a temperature sufficient to provide a glass-like material and adding the glass-like material in to the active cannabinoid.

12. The method of claim 1, wherein the method comprises solubilizing the cannabinoic acid in a carrier to provide a solution and adding the solution to the active cannabinoid.

13. The method of claim 1, further comprising adding a flavor modifier or a fragrance modifier.

14. The method of claim 13, wherein the flavor modifier comprises one or more terpenes.

15. A composition comprising about 1 wt. % to about 60 wt. % of a cannabinoic acid, about 40 wt. % to about 99 wt. % of active cannabinoid, and less than about 2 wt. % of endogenous terpenes.

16. The composition of claim 15, wherein the active cannabinoid is tetrahydrocannabinol (THC).

17. The composition of claim 15, wherein the cannabinoic acid is tetrahydrocannabinolic acid (THCA), cannabidiolic acid (CBDA), cannabinolic acid (CBNA), cannabichromenic acid (CBCA), cannabigerolic acid (CBGA), cannabinodiolic acid (CBNDA), cannabicyclolic acid (CBLA), cannabivarinic acid (CBVA), tetrahydrocannabivarinic acid (THCVA), cannabidivarinic acid (CBDVA), cannabichrovarinic acid (CBCVA), cannabigerovarinic acid (CBGVA), cannabigerol monomethylether (CBGM), cannabigerolic acid monomethylether (CBGAM), or a combination of any two or more thereof.

18. The composition of claim 15, wherein the active cannabinoid has a purity of greater than 95% and/or wherein the cannabinoic acid has a purity of greater than 95%.

19. The composition of claim 15, wherein the active cannabinoid is selected from the group consisting of Δ⁸-tetrahydrocannabinol (Δ⁸-THC), Δ⁹-tetrahydrocannabinol (Δ⁹-THC), Δ¹⁰-tetrahydrocannabinol (Δ¹⁰-THC), or a combination of any two or more thereof.

20. The composition of claim 15, wherein the cannabinoic acid is selected from the group consisting of 2-Δ⁸-tetrahydrocannabinolic acid (2-Δ⁸-THCA), 4-Δ⁸-tetrahydrocannabinolic acid (4-Δ⁸-THCA), 9-Δ⁸-tetrahydrocannabinolic acid (9-Δ⁸-THCA), 2-Δ⁹-tetrahydrocannabinolic acid (2-Δ⁹-THCA), 4-Δ⁹-tetrahydrocannabinolic acid (4-Δ⁹-THCA), 9-Δ⁹-tetrahydrocannabinolic acid (9-Δ⁹-THCA), 2-Δ¹⁰-tetrahydrocannabinolic acid (2-Δ¹⁰-THCA), 4-Δ¹⁰-tetrahydrocannabinolic acid (4-Δ¹⁰-THCA), 9-Δ¹⁰-tetrahydrocannabinolic acid (9-Δ¹⁰-THCA), cannabidiolic acid, and cannabigerolic acid, or a combination of any two or more thereof.

21. The composition of claim 15, further comprising about 1 wt. % to about 98 wt. % of a carrier.

22. The composition of claim 15, further comprising a flavor modifier or a fragrance modifier.

23. The composition of claim 15, comprising about 4 wt. % to about 15 wt. % of a cannabinoic acid, about 40 wt. % to about 99 wt. % of active cannabinoid and about 4 wt. % to about 15 wt. % of a flavor modifier.

24. The composition of claim 22, wherein the flavor modifier comprises one or more terpenes.

25. The composition of claim 24, wherein the one or more terpenes are added exogenously.
